(19) United States
(12) Patent Application Publication
(10) Pub. No.: US 2025/0028049 A1
(43) Pub. Date: Jan. 23, 2025

(54) PATH-INTEGRATED OPTICAL SENSING AND SIMULTANEOUS RETROREFLECTOR TRACKING

(71) Applicant: The Trustees of Princeton University, Princeton, NJ (US)

(72) Inventors: Michael Soskind, Plainsboro, NJ (US); Felix Kleemann, Lower Saxony (DE); Gerard Wysocki, Princeton, NJ (US)

(73) Assignee: The Trustees of Princeton University, Princeton, NJ (US)

(21) Appl. No.: 18/778,223

(22) Filed: Jul. 19, 2024

Related U.S. Application Data
(60) Provisional application No. 63/527,724, filed on Jul. 19, 2023.

(51) Int. Cl. G01S 17/66; G01N 21/31; G01N 33/00; G01S 7/481

(52) U.S. Cl. CPC G01S 17/66; G01N 21/31; G01N 33/0027; G01S 7/4814; G01S 7/4816; G01S 7/4817; G01N 2201/06113; G01N 2201/10

(57) ABSTRACT

A retroreflector tracking and sensing technique is provided. Systems may include a laser source, optical component(s), and a beam steering component disposed along an optical transmit path. The optical component(s) may be disposed between the laser source and the beam steering component (such as a mirror coupled to an actuator). The systems may include a position-sensitive detector (PSD). The beam steering component, one or more of the optical component(s), and the PSD may be disposed along an optical return path. The optical component(s) in the optical return path may be disposed between the beam steering component and the position-sensitive detector. The PSD may be operably coupled to a position tracking analyzer and an optical sensing analyzer. Electrical signals generated by the PSD may be configured to be used for retroreflector tracking and/or optical sensing.

### CROSS-REFERENCE TO RELATED APPLICATIONS

The present application claims priority to U.S. Provisional Patent Application No. 63/527,724, filed Jul. 19, 2023, the contents of which are incorporated by reference herein in its entirety.

### TECHNICAL FIELD

The present disclosure is drawn to technique for optical sensing and tracking, and in particular, techniques involving simultaneous optical sensing and retroreflector tracking.

### BACKGROUND

Methane (CH₄) has grown in interest recently due to the prominent use of natural gas in power-to-X (PTX) energy solutions. CH₄ has a global warming potential of 80-83 times that of CO₂ over 20-year timescales and is flammable and potentially dangerous at high concentration. Emissions of greenhouse gases such as CH₄ can therefore have a major impact on global climate change, human health, and efficiency of PTX energy transmission. Other than CO₂ emissions which are closely tied to combustion processes, methane has a broad variety of sources and is strongly delocalized. It is known that the EPA methane emissions inventory vastly underrepresents the true emissions of methane, both in the natural gas sector as well as wastewater sector. In particular, these emissions are likely primarily caused by superemitter events, which account for more than half of the natural gas emissions. Therefore, it is important to accurately locate and quantify fugitive leaks along the natural gas infrastructure.

Several systems have been shown to effectively localize methane emissions. One promising technique that was recently introduced was the use of an open-path spectroscopic sensor that actively tracked a drone in order to map an estimated plume source location as well as emission rate. The system showed that the source could be estimated to within 1 meter of its known position, and was able to estimate the release to within +30% error with respect to the low flow rate release. That system used a sensitive spectroscopic technique known as chirped laser dispersion spectroscopy (CLaDS), which proves to be highly linear and precise, but requires expensive high-speed detectors and complicated radio-frequency (RF) electronics.

Indeed, conventional solutions to the challenge of alignment and pointing and tracking incorporate additional detectors and electronics, and thereby introduce further cost and complexity to the systems.

### BRIEF SUMMARY

In various aspects, a retroreflector tracking and sensing system may be provided. The system may include a laser source (such as a semiconductor laser), one or more optical components, and a mirror coupled to a beam steering actuator disposed along an optical transmit path. The optical components may be disposed between the laser and the mirror coupled to the beam steering actuator. The system may include a position-sensitive detector. The mirror coupled to the beam steering actuator, at least one of the optical components, and the position-sensitive detector may be disposed along an optical return path. The at least one of the optical components may be disposed between the mirror coupled to the beam steering actuator and the position-sensitive detector. The position-sensitive detector may be operably coupled to a position tracking analyzer and an optical sensing analyzer. Electrical signals generated by said position-sensitive detector may be configured to be used for retroreflector tracking and/or optical sensing.

The system may include a retroreflective target forming an end of the optical transmit path and a beginning of the optical return path. The retroreflective target may be mounted on, e.g., a manned or unmanned vehicle, such as an unmanned aerial vehicle (UAV), a blimp, a ground vehicle (such as a car, motorcycle, truck, personnel carrier, etc.), a boat, a helicopter, or may be mounted on, e.g., a fixed object, such as an outdoor fixed object.

In certain aspects, system may be fixed, or mobile. In certain aspects, the retroreflective target may be mobile, and the position-sensitive detector, the laser source, the optical component(s), and the mirror coupled to the beam steering actuator may be stationary. In certain aspects, the retroreflective target may be stationary, and the position-sensitive detector, the laser, the optical components, and the mirror coupled to the beam steering actuator are mobile.

The optical components may include a first optical component with positive optical power disposed in both the optical transmit path and the optical return path. The system may include at least one additional component with optical power between the first optical component and the position-sensitive detector. The system may include a beam splitter disposed in both the optical transmit path and the optical return path, between the laser source and the at least one of the optical components (i.e., the optical component(s) common to both the transmit and return paths).

The system may include an intermediate mirror coupled to an additional beam steering actuator. This additional mirror may be disposed in both the optical transmit path and the optical return path. This additional mirror may be disposed between, e.g., a beam splitter and the at least one of the optical components.

The system may include one or more additional optical element(s) disposed only in the optical return path between the beam splitter and the position-sensitive detector. Thus, the system may include 1) one or more optical element(s) that are in both the transmit and return path, and 2) optionally may include (a) optical elements that are only in the transmit path, (b) optical elements that are only in the return path, or (c) both (a) and (b).

The system may include a mirror disposed in the optical transmit path between the laser and the beam splitter. The optical component(s) may include an optical circulator in both the optical transmit path and the optical return path. The optical circulator may be composed of a waveplate and a polarizing beam splitter in both the optical transmit path and the optical return path. The polarizing beam splitter may redirect laser light from the laser source into the optical transmit path and the return light in the optical return path towards the position-sensitive detector.

In certain aspects, the at least one of the optical components defines a telescope disposed in both the optical transmit path and the optical return path. The telescope may be disposed between a waveplate and the mirror coupled to the beam steering actuator.

In various aspects, a method for collecting tracking and optical sensing data simultaneously using a retroreflector tracking and sensing system may be provided. The method may include directing a collimated laser beam from a laser along an optical transmit path, using at least a mirror coupled to a beam steering actuator. The method may include directing return light towards optical elements along an optical return path using at least the mirror coupled to the beam steering actuator. The method may include directing the return light received by the optical elements towards a position-sensitive detector. The position-sensitive detector may be being operably coupled to a position tracking analyzer and an optical sensing analyzer. Information generated by the position-sensitive detector may be configured to be used for retroreflector tracking and/or optical sensing.

The method may include controlling the beam steering actuator based on a determination by the position tracking analyzer. The method may include extracting optical sensing data from the information generated by said position-sensitive detector with the optical sensing analyzer. The method may include extracting spectroscopic data from the information generated by said position-sensitive detector with the optical sensing analyzer.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a general schematic showing one embodiment of a system.

FIG. 2 is a general schematic showing a second embodiment of a system.

FIGS. 3A and 3B are general schematics showing a third embodiment of a system used for both spectroscopic sensing of, e.g., a gas (3A) and drone tracking (3B).

FIG. 4 is a general schematic showing a fourth embodiment of a system.

FIG. 5 is a general schematic showing a fifth embodiment of a system.

FIG. 6 is a graph showing an example of the system without wavelength-locking enabled, compared to with wavelength locking switched on, over 10 second duration. Inset is the 3f signal and operating region used for locking the frequency.

FIG. 7 is a graph showing an example of flow-based measurements with good agreement at low concentrations and some deviation at higher concentrations due to absorption saturation.

FIG. 8 is a graph showing an example of drone position overlaid with measured methane concentration, demonstrating system capability to track and accurately measure methane concentration of a moving object.

FIG. 9 is a graph showing a linear relation between distance and extracted concentration. Finding such a relationship allows for calibrating the optical system performance.

FIG. 10 are graphs showing the Allan variance metric of the system calculated for some of the data during the stationary portion of a drone flight, showcasing a precision of 0.77 ppm-m/Hz^{1/2}.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

Disclosed herein are a device and a method for path-integrated optical sensing and simultaneous retroreflector tracking using a multi-functional position-sensitive photodetector (PSD).

More particularly, disclosed are a system and a method for simultaneously measuring optical/spectroscopic signals in a retroreflector-based remote sensing configuration, while enabling simultaneous active tracking of the retroreflecting target. The disclosed approach leverages a single PSD and can be implemented with a variety of existing optical open-path sensing techniques, enabling new applications in remote sensing, as well as reduced system complexity, size, weight, and cost. A proof-of-concept demonstration was performed for the purpose of drone-assisted detection of atmospheric methane using the wavelength modulation spectroscopy (WMS) technique and a position-sensitive quadrant photodetector (QPD), which is a particular type of PSD.

The disclosed approach has utility, inter alia, as follows:

A) The simultaneous laser beam pointing and tracking performed together with spectroscopic and/or optical sensing is applicable to many optical systems that interrogate a retroreflector-based open-path configuration for path-integrated sensing (e.g., spectroscopic sensing of trace-gases, distance detection etc.). The method can account for rapid and significant errors in the pointing angle of the laser system and correct for these errors with the integration of one's choice of control scheme. This allows for robust operation in harsh environments and outdoor settings with minimal human intervention.

B) The method can be incorporated to track a variety of retroreflector-carrying mobile vehicles, transportation systems, or stationary objects, including but not limited to: drones/UAVs, blimps, automotive vehicles, boats, tanks, helicopters, and outdoor reflectors fixed to moving or unstable objects. Alternatively, if the system is mounted on a moving vehicle, it can track stationary retroreflector targets, or other retroreflector-carrying mobile vehicles.

C) These types of systems could aid pointing and tracking challenges associated with spectroscopy (or any other optical sensing techniques) at long distances induced by atmospheric turbulence, by maintaining optical power on the detector throughout measurement time. Sustaining high optical power returns in fluctuating conditions is crucial for achieving high signal to noise ratios in optical sensing systems, and this could be an enabling method for correcting for fluctuations caused by a variety of factors external to the system.

D) The technique can be extended to systems that have fixed mirrors or reflectors as a self-aligning and calibrating instrument for field applications. Alignment errors during system transport in fixed systems can be corrected automatically by incorporating this technique, requiring less field servicing of instruments.

E) The fields of use for a system such as this include, but are not limited to trace gas and atmospheric sensing, chemical detection of explosive devices, structural health monitoring, distance measurements, etc.

The disclosed technique enables measurement capabilities similar to an earlier demonstrated open-path spectroscopic sensor that can actively track a drone in order to map an estimated plume source location as well as emission rate. That system used a sensitive spectroscopic technique known as chirped laser dispersion spectroscopy (CLaDS), which offers the advantages of highly linear and precise methane emission monitoring. See, e.g., U.S. Pat. No. 10,656,083. The CLaDS-based system demonstrated methane localization to within 1 meter accuracy and was able to estimate leak concentrations to +30% error at a release rate more than an order of magnitude smaller than rates typically observed at natural gas facilities. However, the CLaDS technique requires expensive high-speed detectors and advanced radio-frequency electronics, making systems based on CLaDS significantly more costly. Furthermore, the CLaDS technique uses a detector that is very small, requiring precise pointing at the retroreflector, and leading to data loss due to alignment issues. Moreover, to enable tracking of moving objects, a separate PSD had to be used in addition to a fast-photodetector specifically used for acquiring the CLaDS measurement signals. While CLaDS equipped with a separate retroreflecting tracking sub-system has shown good performance in drone-based detection of small methane leaks, many applications could benefit from alternative techniques that provide robust field operation and require hardware that is less expensive, less complicated, and less sensitive to misalignment.

The system disclosed here aims to significantly reduce the overall cost, complexity, and size of a sensing system capable of real-time retroreflector tracking, while providing similar performance and functionality to the CLaDS equipped with retroreflector tracking system. The disclosed system leverages a conventional, line-locked WMS spectroscopic sensing technique for open-path detection of methane. An important feature is the use of a single, dual-purpose quadrant photodetector to perform both spectroscopic sensing and active tracking of a retroreflecting target (demonstrated with a flying drone). Additionally, the system may optionally use a fiber-coupled reference arm for WMS signal calibration and wavelength-locking (but such may be considered non-mandatory diagnostic features). The system aims to be a cost-effective, easily deployable solution for precise detection of trace gases (e.g., methane leaks) and can perform any other form of optical sensing provided that the bandwidth of the PSD is sufficient for the application. The techniques discussed herein can be utilized at many other wavelengths in order to probe alternative spectroscopic transitions or be used with different types of optical sensing techniques. The use of multi-functional PSDs enables new and agile methods for remote optical detection.

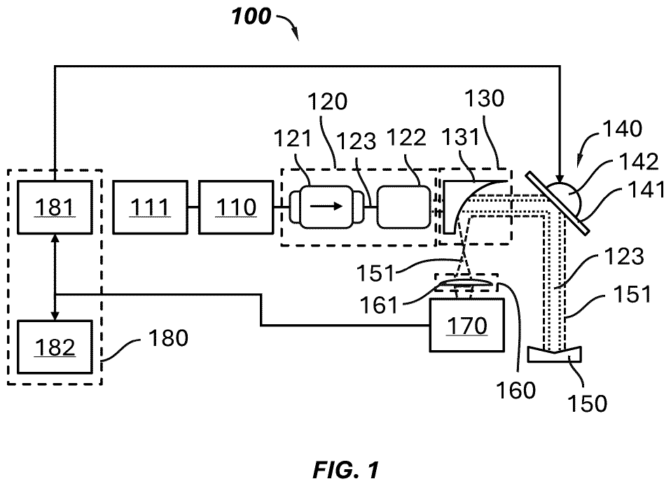

In various aspects, a retroreflector tracking and sensing system may be provided. Referring to FIG. 1, the system (100) may include a laser source (110). The laser may be any appropriate laser source, such as a semiconductor laser, including a laser diode within a semiconductor package.

Broadly, the systems are generally configured to use the laser source (110) to create an optical transmit path (123) which is directed, using at least one controllable mirror (e.g., mirror (141)), towards a retroreflective target (150). An optical return path (151) is then formed back towards the controllable mirror, which is eventually directed back to a position-sensitive detector (170) (PSD). The PSD may provide two sets of signals: one for position tracking and a second for optical (or in one example case, spectroscopic) sensing.

The laser source (110) may be operably coupled to, e.g., a controller (111) configured to control the output of the laser source. As used herein, the term “controller” refers to a hardware device or a software that manages, commands, directs, or regulates operation of other components, devices, or systems. This may include not only those integrated circuits typically referred to in the art as a controller, but may also include a processor, a microprocessor, a microcontroller, a programmable logic controller, an application specific integrated circuit, and/or other programmable circuitry.

Light from the laser source may pass through one or more optical components. As used herein, the term “optical component” generally refers to optical elements, such as optical waveguides, optical fibers, lenses, gratings, prisms, filters, collimators, and so forth. For clarity, as used herein, mirrors are considered distinct from optical components.

The optical components may include one or more portions. There may be an optical component disposed within the optical transmit path (123) but not within the optical return path (151); such a component may be referred to as transmit optical componentry (120). There will generally always be an optical component disposed within both the optical transmit path (123) and within the optical return path (151); such a component may be referred to as transmit/return optical componentry (130). There may be an optical component disposed within the optical return path (151) but not within the optical transmit path (123); such a component may be referred to as return optical componentry (160).

As noted, there will always be at least one transmit/return optical componentry (130). There may optionally be one or more transmit optical components and/or one or more return optical components. In some embodiments, the system may be free of any transmit optical componentry (120). In some embodiments, the system may include one or more transmit optical componentry (120). In some embodiments, the system may include a plurality of transmit optical componentry (120). In some embodiments, the system may be free of any return optical componentry (160). In some embodiments, the system may include one or more return optical componentry (160). In some embodiments, the system may include a plurality of return optical componentry (160).

In FIG. 1, one embodiment is disclosed, the transmit optical componentry (120) is shown as including an amplifier (121) and a collimator (122). In FIG. 1, the transmit/return optical componentry (130) is shown as including an off-axis parabolic lens (131) (OAP). In FIG. 1, the return optical componentry (160) are shown as including a lens (161).

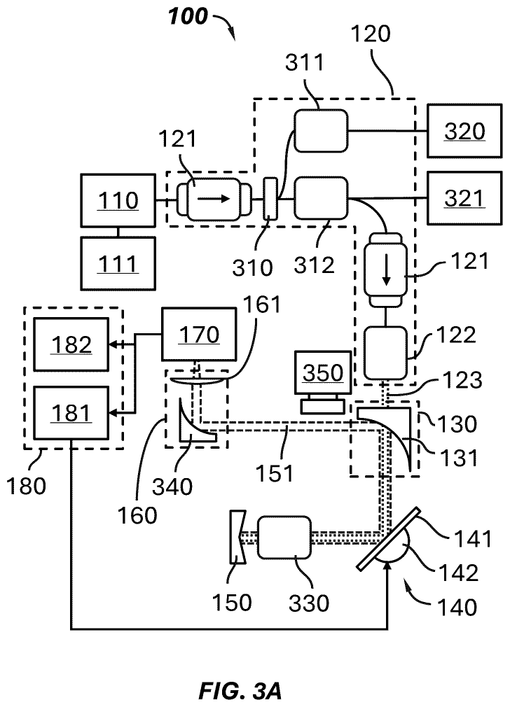

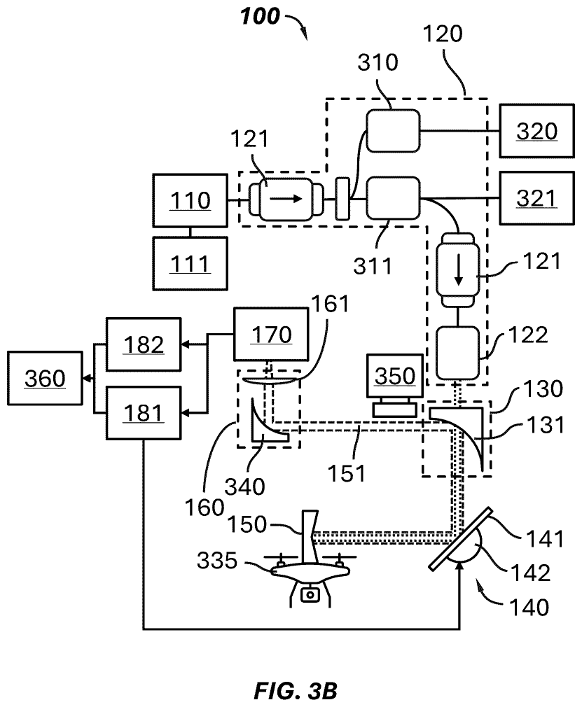

The optical componentry may include optical isolators, as can be seen in FIG. 3A, which can be useful in laser systems to minimize the effect of interference effects induced by back reflection of laser radiation into the laser cavity. Other optics, such as fiber based, chip based, or free space optical components can be used in the various systems.

The system may include a beam steering component (140). The beam steering component may be a mirror (141) operably coupled to a beam steering actuator (142) disposed along an optical transmit path. The beam steering actuator may allow, e.g., control of the orientation of the mirror in around multiple axis, such as around two axes, such as around an elevation (or altitude) axis and an azimuth axis.

As shown in FIG. 1, some optical components (e.g., transmit optical componentry (120) and transmit/return optical componentry (160)) may be disposed between the laser source (110) and the mirror (141) coupled to the beam steering actuator (142). Thus, the optical transmit path (123) may be configured to originate with the laser source (110), pass through optical components (e.g., any transmit optical componentry (120) and the transmit/return optical componentry (130)), then be directed by the beam steering component (140) towards a target.

The system may include a position-sensitive detector (170) configured to detect light returned to the system. An optical return path may thus include light received and reflected from the beam steering component (140) towards at least some of the optical components in the optical transmit path (e.g., the transmit/return optical componentry (130)), and may optionally pass through additionally optical components (e.g., any return optical componentry (160) towards a detector. Said differently, the mirror (141) coupled to the beam steering actuator (142), at least one of the optical components, and the position-sensitive detector (170) may be disposed along an optical return path. Such optical components on the optical return path may be disposed between the mirror coupled to the beam steering actuator and the position-sensitive detector.

Furthermore, while these figures shown a mechanical steering actuator for manipulating the beam steering component, non-mechanical beam steering, such as that capable with tiled aperture laser arrays, may be utilized as an alternative beam steering mechanism for fine pointing control.

As used herein, the term “position-sensitive detector (PSD)” refers to an optoelectronic sensor that typically utilizes photodiode surface resistance to measure the position of the integral focus of an incoming light signal by converting a light spot on the sensor surface into an electrical signal corresponding to the focal position of the light spot. The PSD may include, e.g., one-dimensional, or may be multi-dimensional (e.g., 2-dimensional or 3-dimensional) sensors. The PSD may include any known PSD, including a photodiode or photodiode array, a complementary metal-oxide semiconductor (CMOS) sensor, a charge-coupled device (CCD), etc. In one preferred embodiment, the PSD is a quadrant photodiode (QPD).

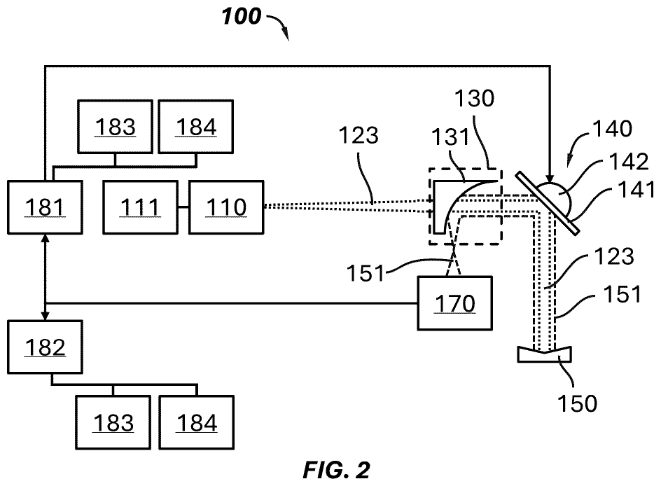

The position-sensitive detector may be operably coupled to one or more devices configured to performing position tracking and optical sensing. In FIG. 1, the system is shown as including a processing unit (181) configured as a position tracking analyzer, and a processing unit (182) configured as an optical sensing analyzer. These functions may be performed by a single device (180) or multiple devices. Each function may be performed by one or more processing units. Each processing unit(s) may, independently, be operably coupled to associated memory (183, see FIG. 2) and a non-transitory computer readable storage device (184, see FIG. 2) containing instructions that, when executed by its associated processing unit(s), performs specific tasks to accomplish the desired functions. As will be understood, these functions (position tracking and optical sensing) may be performed by separate devices, or there may be a single device configured to perform both functions. In FIG. 1, the two functions are performed by the Electrical signals generated by said position-sensitive detector may be configured to be used for retroreflector tracking and/or optical sensing.

The system may include a retroreflective target (150) forming an end of the optical transmit path (123) and a beginning of the optical return path (151). The retroreflective target may be mounted on a device, which may be stationary or mobile. For example, the retroreflective target may be mounted on, e.g., a manned or unmanned vehicle, such as an unmanned aerial vehicle (UAV), a blimp, an automotive vehicle, a boat, a helicopter, or may be mounted on, e.g., a fixed object, such as an outdoor fixed object (e.g., an exterior surface of a building, an outdoor sign, a storage tank or vessel, etc.).

As will be appreciated, the arrangement of components in FIG. 1 is not limiting, and other variations are readily envisioned.

Referring briefly to FIG. 2, in some embodiments, no transmit optical componentry (120) or return optical componentry (160) are present. In FIG. 2, a collimated laser beam from the laser source is sent towards the target (here, retroreflective target (150) via a beam steering component (140) as disclosed herein, and after reflection from the retroreflecting target, the system redirects the return light towards the PSD via an off-axis parabolic lens. The position-sensitive signal from the PSD for tracking is used in an active feedback loop to control the beam steering actuator to maintain the alignment of the laser to the retroreflecting target.

Referring to FIGS. 3A and 3B, another embodiment of a system is provided, showing how such systems can be used for both tracking and optical (or spectroscopic) sensing. In FIG. 3A, the transmit optical componentry (120) includes an amplifier (121) prior to an optical splitter (310). The optical splitter may be, e.g., a 90:10 splitter, an 80:20 splitter, etc. A first output from the splitter feeds into a first gas cell (311) and a first photodetector (320). A second output from the splitter feeds into a second gas cell (312) and then splits again, to a second photodetector (321) and another amplifier (121) before being passed to a collimator (122).

In FIG. 3A, after passing through the transmit/return optical componentry (130), the collimated light is reflected by the beam steering component (140) towards a volume of space (330) containing a gas (such as a sample gas cell). At least some of the light travels through the volume of space to the retroreflective target (150), where it then passes back through the volume of space along the optical return path (151).

In FIG. 3B, after passing through the transmit/return optical componentry (130), the collimated light is reflected by the beam steering component (140) towards a mobile target (here, drone (335)) and specifically to the retroreflective target (150) operably coupled to the mobile target, where it then gets reflected back towards the beam steering component (140) along the optical return path (151), before further directed towards the detector by the off-axis parabolic lens (131)

In FIGS. 3A and 3B, the return optical componentry (160) includes an additional off-axis parabolic lens (340) and a lens (161).

As shown in FIGS. 3A and 3B, the system may include a camera (350). The camera may be operably coupled to one or more processing unit(s) (such as processing unit (181) configured as a position tracking analyzer, and/or processing unit (182) configured as an optical sensing analyzer). In this manner, the camera may provide additional data for the various analyses to utilize.

As shown in FIG. 3B, in some embodiments, the optical sensing analyzer and the position tracking analyzer may transmit data to a remote processing unit (360). For example, the data may be sent to a remote server to store any collected data.

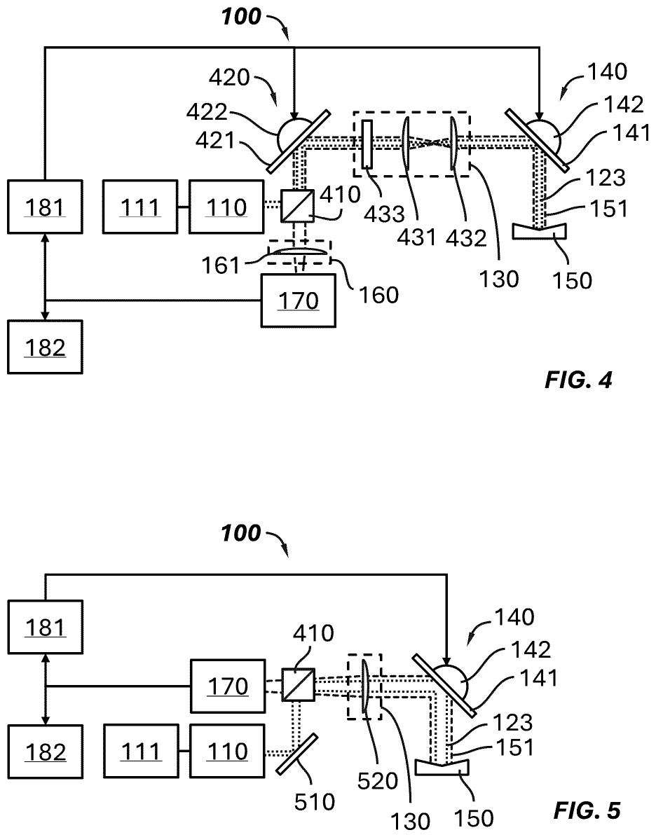

FIG. 4 shows an alternative schematic for achieving simultaneous position tracking and optical sensing. Collimated laser light is reflected from a beam splitter (410) and first beam steering actuator (420) towards transmit/return optical componentry (130). The transmit/return optical componentry (130) can include a variety of components, including lenses (431, 432) such as those comprising a telescope, as well as polarization dependent components (433), such as a waveplate, which modify the radiation and direct the laser light towards the target via an optional second beam steering component (140). After reflection from the retroreflecting target, the beam steering components (140, 420) redirects the return light towards the PSD (170) via the same transmit/return optical componentry (430). The position-sensitive signal from the PSD for tracking is used in an active feedback loop to control the beam steering actuator to maintain the alignment of the laser to the retroreflecting target. This primary beam steering actuator can also take the form of fixed mirror with steering performed using non-mechanically steered laser radiation, for example through the use of a tiled aperture array.

FIG. 5 shows an alternative schematic for achieving simultaneous position tracking and optical sensing. Divergent laser radiation is collimated via the transmit/return optical componentry (130) after reflection from a beam splitter (410), and directed towards the target via a beam steering component (140), in this case using a single steering mirror (141). After reflection from the retroreflective target (150), the beam steering component (140) redirects the return light towards the PSD (170) via the same transmit/return optical componentry (130). The position-sensitive signal from the PSD for tracking is used in an active feedback loop to control the beam steering actuator to maintain the alignment of the laser to the retroreflecting target.

### Example

As noted previously, it is important to accurately locate and quantify fugitive leaks along the natural gas infrastructure. This example study was performed by implementing spectroscopic remote detection of methane.

The study used a system with the configuration shown in FIG. 3A. The light source was a laser diode (NEL NLKIUSEIAA, LD) operated at 1653.7 nm, in which the radiation was split into two fiber-coupled reference paths for line-locking and concentration reference, as well as one free space path for drone tracking and concentration measurement. For line-locking, a 20% methane, 3 cm gas cell (Wavelength Reference CH₄/N₂-H(3)-20%/740-FCAPC, e.g., first gas cell (311)) and a photodetector (Thorlabs DET01CFC, e.g., first photodetector (320)) were used. The signal was split and fed into a DAQ-Card (NI USB-6259, DAC) and a hardware Lock-In Amplifier (Femto LIA-MV-200-H) (e.g., processing units (181, 182)) for line-locking to the third harmonic using a hardware proportional-integral-derivative (PID) controller (SRS SIM960). A second methane reference cell at 1% and 3 cm in length (Wavelength Reference CH4/N2-H(3)-1%/740-FCAPC, e.g., second gas cell (312)) was used to measure a reference concentration using a gain-adjustable photodetector (Thorlabs PDA10CS, e.g., second photodetector (321)). The gas cell also provides an offset to the measurement performed by the quadrant photodetector (Thorlabs PDQ30C, e.g., position-sensitive detector (170)) that is used as the main, multi-functional photodetector in the prototype system. The QPD performs two distinct functions: 1) its position-sensing capability is utilized to perform the retroreflector tracking, while 2) its integrated four-quadrant signals are also processed to extract spectroscopic information associated with the open-path trace-gas measurement. The detected signals are acquired by the DAC and demodulated at the first and second harmonics used to acquire the spectroscopic signal of methane in the open-path sensing configuration. The digital lock-in amplifiers (LIAs) are implemented in National Instruments LabVIEW software onboard the DACs and used for the measurements demonstrating the system capabilities. The QPD provides X and Y error signals used for tracking the drone-mounted retroreflector (RR) spatially, and the tracking is performed using a motorized gimbal mirror (Acrotech AMG150GR, e.g., beam steering component (140)).

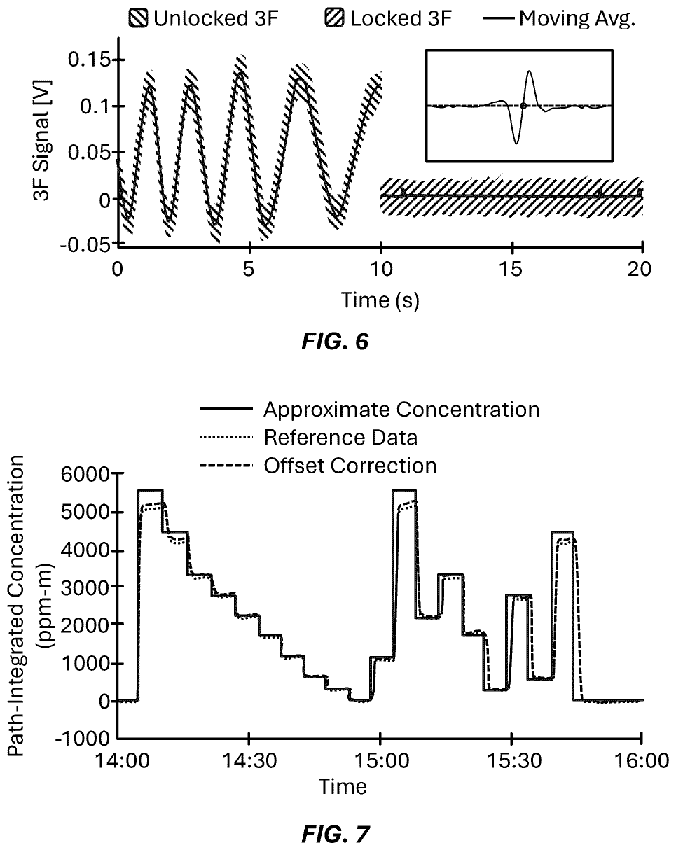

A number of techniques have been proposed in the past for wavelength stabilization in WMS systems. The example system uses line-locking electronics with a dedicated hardware LIA and PID controller to improve the central wavelength stability, as shown in FIG. 6. The figure compares the 3f signal in the unlocked state and the locked state across two different 10 second measurements. By reducing the low frequency drift present in the wavelength of the laser as shown in the unlocked case, the 2f signals also have reduced measurement error caused by a changing wavelength during acquisition and increase the long-term stability of the measurement system. In the inset of FIG. 6, an experimentally derived 3f signal is shown with a circle at the zero-crossing point used for locking with the current feedback loop for illustrative purposes. By actively controlling the laser operating current, the system mitigates drift induced by wavelength fluctuation. To accurately estimate the 2f/1f normalized signal, the gain adjustable photodetector (in this example, second photodetector (321) in FIG. 3A) is used as a reference detector, while the QPD (in this example, PSD (170)) is used for measuring the open-path CH₄ concentration. However, it should be noted that this approach was implemented in the example system as a form of diagnostics, and both frequency stabilization as well as signal amplitude referencing can also be performed without the need for additional photodetectors (with only a single PSD).

The use of a cost-effective, off-the-shelf QPD for both object-tracking and spectroscopic measurement during drone flight has been successfully demonstrated. QPDs have been used in laser beam pointing and tracking systems for a variety of applications, particularly free-space optical communications systems (both traditional and quantum). Typically, quadrant detectors are used in conjunction with fast steering mirrors or galvanometers for applications with limited field of regard. In this system, a gimbal mirror was used as the main steering component. The gimbal mirror limits the bandwidth of the tracking system, while at the same time allowing for a significant increase in beam steering range and therefore field of regard of the scene, with up to 360-degree coverage for appropriate applications. A proportional-integral (PI) control method provides real-time tracking of a mobile retroreflector, and the tracking system has been previously described on a spectroscopic system.

The presently described system has the main advantage of using the QPD signal for both the mobile retroreflector tracking and, e.g., CH₄ trace-gas sensing. Because the QPD provides both capabilities, the receiver optics can be significantly simplified and continuous acquisition of spectroscopic information during tracking can be achieved. The simplified receiver architecture removes the added requirement of careful co-alignment between two different detectors (signal detector and secondary QPD). More importantly the significantly larger size of the QPDs increases the object field of view of the spectroscopic detector in comparison to, e.g., the CLaDS-based systems discussed previously.

To measure the linearity of concentration extraction using line-locked 2f/1f WMS using the QPD, the concentration of a gas cell placed in front of the retroreflector was modified using a gas-mixing flow system (Alicat, M-Series). The path-integrated concentration measurement shown in FIG. 7 demonstrates the signal-concentration linearity by sending the free-space path through a gas flow cell and regulating the mixture of the flowing gas. The gas flow regulation system mixes Air (Airgas, Industrial Grade Air) with CH₄ (NORCO, 2.5±0.05%) to change the methane concentration in the flow cell, therefore affecting the path-integrated concentration. The CH₄ path-integrated concentration ranges from 10 ppm-m with air flowing through the cell, all the way up to 5500 ppm-m with the cell full of 2.5% CH₄ in air. FIG. 7 displays the experimental results as well as the expected path-integrated concentrations based on the controlled flow rates. The methane concentration is measured by 2f/1f normalizing all signals and comparing it directly to the gain adjustable photodetector (in this example, second photodetector (321) in FIG. 3A) reference detector 2f/1f signal (“reference data”, dotted line) or by additionally correcting for the estimated offset induced in the free-space path by the second gas cell (e.g., second gas cell (312) in FIG. 3A) in the system by correlating first photodetector (320) data to that from second photodetector (321) (“offset correction”, dashed line). Good agreement between theoretical and measured concentrations <4000 ppm-m was observed. Slight discrepancies at higher concentrations (corresponding to peak absorbances of 14% or higher) are visible and are expected due to the exponential character of the Beer-Lambert law. Significant nonlinearity in trace gas concentration measurements associated with natural gas leaks at facility scale (up to a few hundred meters) are not anticipated, and if moving to longer distance sensing, alternative absorption lines with reduced line-strength can be easily considered. The disclosed system offers a promising solution in applications such as natural gas facilities monitoring, particularly because using agile systems such as drone-based techniques can reduce sensing time and improve coverage of the facility. Fixed transceiver-based methane sensing techniques have been proposed recently and have shown promise in localizing low flow-rate controlled releases.

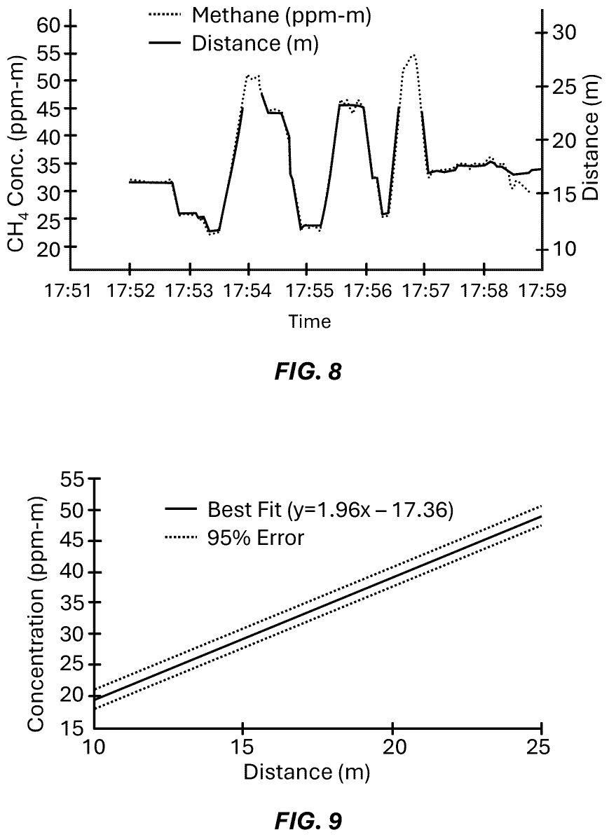

Demonstration of the simultaneous tracking of a drone-mounted mobile retroreflector and methane concentration retrieval method is shown in FIG. 8. The position of drone-mounted RR changes the absolute path length between the drone and the optical system. The drone was flown safely in a controlled laboratory approved for drone flight. The position of the retroreflector and the gimbal mirror are tracked and localized by using an external infrared tracking system (VICON) to correlate the measured 2f/1f-based concentration estimate to a theoretical estimate of expected concentration based on the path length. The VICON system allows for the drone and system positions to be measured with sub-centimeter precision. The direct correlation between the path length and measured CH₄ concentration are shown for distances less than 12.5 meters (25 meters round-trip path) in FIG. 8. Measured methane concentrations at which there are not measured distances indicate that the VICON system was unable to triangulate the object positions and orientations at those locations.

In order to improve the measured concentration accuracy with the WMS system, a calibration technique was implemented. The concentration and distance data, displayed in FIG. 8, demonstrate very good linearity with respect to one another throughout the duration of the drone tracking measurement. In the case of a well-mixed room, the measurement of methane concentration using a calibrated gas measurement system should match that measured with the open-path WMS system. Using a LICOR LI-7810, the methane concentration measured in the room was 2.03 ppm.

By plotting the data from the measurement in FIG. 8, with the distance on the x-axis and the path-integrated methane concentration on the y-axis, a linear regression was performed (see FIG. 9) to find the linear function with minimum square error. The slope represents the extracted concentration within the environment, while the offset value corresponds to a measured error induced by the concentration within the fiber-coupled gas cell (second gas cell (312)). Based on an estimate, the concentration within the 1% gas cell is closer to 0.94%. The data in FIG. 8 shows very good agreement throughout the flight duration between distance and concentration.

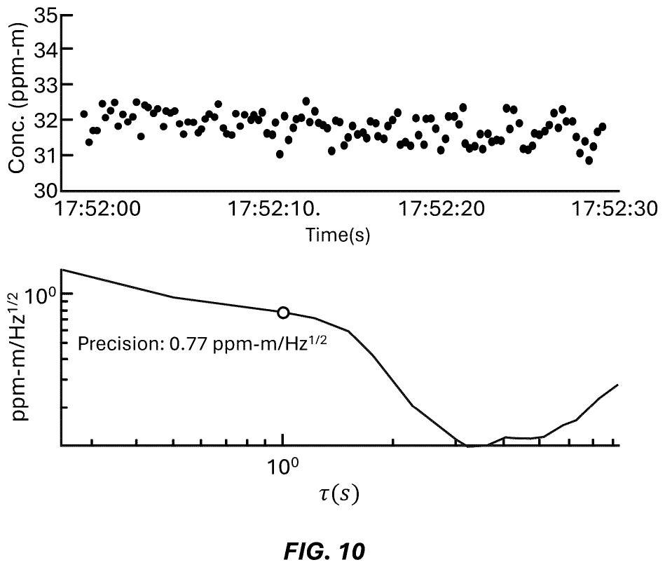

An Allan variance plot for the system measuring the concentration during drone flight is shown in FIG. 10. Taking one of the positions in which the system hovered for approximately 30 seconds, the Allan variance analysis was performed, which indicates a precision of 0.77 ppm-m/Hz^{1/2} with 1 second averaging time, and is indicated by the open circle on the bottom plot. This is a relatively short duration measurement that can only indicate short-term performance of the system, but it already demonstrates the potential of the system for high precision concentration measurements.

The accuracy was confirmed by using a reference measurement system (LICOR LI-7810) that provided an average methane concentration of 2.03 ppm, and the prototype system estimates methane concentration of 1.96 ppm which is only 3.5% lower than the ground truth. It is anticipated that the relative simplicity of this disclosed system will enable future applications of smaller, lower-cost, and more sensitive sensing systems.

The disclosed spectroscopic system uses less expensive reference detectors and enables real-time sensing of, e.g., methane concentration. The system also shows the ability to sense mobile objects, without a loss in precision. Furthermore, by implementing a method for calibration, the system could be fielded for accurate measurement of methane, particularly for monitoring open areas within natural gas facilities or enteric emissions. The system can also determine the optical path length via the phase shift of the 2f signal. For alternative applications looking to perform high-concentration gas sensing, weaker absorption lines could be targeted to reduce non-linearity at high concentrations. Other alternatives to minimize the issue of non-linearity is to use WMS with TDLAS and leveraging a model that can account for absorption at high CH₄ concentrations, or alternatively sensing with a highly linear method such as dispersion spectroscopy.

Various modifications may be made to the systems, methods, apparatus, mechanisms, techniques, and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

What is claimed:

1. A retroreflector tracking and sensing system comprising:
a laser source; one or more optical components, and a beam steering component disposed along an optical transmit path, the one or more optical components being disposed between the laser source and the beam steering component; and
a position-sensitive detector, where the beam steering component, at least one of the one or more optical components, and the position-sensitive detector are disposed along an optical return path, the at least one of the one or more optical components being disposed between the beam steering component and the position-sensitive detector;
wherein the position-sensitive detector is operably coupled to a position tracking analyzer and an optical sensing analyzer, and wherein electrical signals generated by said position-sensitive detector are configured to be used for retroreflector tracking and/or optical sensing.

2. The retroreflector tracking and sensing system of claim 1, wherein the laser source is a semiconductor laser.

3. The retroreflector tracking and sensing system of claim 1, further comprising a retroreflective target forming an end of the optical transmit path and a beginning of the optical return path.

4. The retroreflector tracking and sensing system of claim 3, wherein the retroreflective target is mounted on an unmanned aerial vehicle (UAV), a blimp, a ground vehicle, a boat, a helicopter, or an outdoor fixed object.

5. The retroreflector tracking and sensing system of claim 3, wherein the retroreflective target is mobile, and the position-sensitive detector, the laser source, and the one or more optical components are stationary.

6. The retroreflector tracking and sensing system of claim 3, wherein the retroreflective target is stationary, and the position-sensitive detector, the laser source, the one or more optical components, and the beam steering component are mobile.

7. The retroreflector tracking and sensing system of claim 1, wherein the one or more optical components comprise a first optical component with positive optical power disposed in both the optical transmit path and the optical return path.

8. The retroreflector tracking and sensing system of claim 7, further comprising at least one additional component with optical power between the first optical component and the position-sensitive detector.

9. The retroreflector tracking and sensing system of claim 1, further comprising a beam splitter disposed in both the optical transmit path and the optical return path, between the laser source and the at least one of the one or more optical components.

10. The retroreflector tracking and sensing system of claim 9, further comprising an intermediate beam steering component disposed in both the optical transmit path and the optical return path, between the beam splitter and the at least one of the one or more optical components.

11. The retroreflector tracking and sensing system of claim 10, further comprising additional optical elements disposed only in the optical return path between the beam splitter and the position-sensitive detector.

12. The retroreflector tracking and sensing system of claim 10, further comprising a mirror disposed in the optical transmit path between the laser source and the beam splitter.

13. The retroreflector tracking and sensing system of claim 12, wherein the one or more optical components include an optical circulator in both the optical transmit path and the optical return path.

14. The retroreflector tracking and sensing system of claim 13, wherein the optical circulator is composed of a waveplate and a polarizing beam splitter in both the optical transmit path and the optical return path.

15. The retroreflector tracking and sensing system of claim 14, wherein the polarizing beam splitter directs laser light from the laser source into the optical transmit path and light returning along the optical return path towards the position-sensitive detector.

16. The retroreflector tracking and sensing system of claim 14, wherein the at least one of the one or more optical components defines a telescope disposed in both the optical transmit path and the optical return path.

17. The retroreflector tracking and sensing system of claim 16, wherein the telescope is disposed between the waveplate and the beam steering component.

18. A method for collecting tracking and optical sensing data simultaneously using a retroreflector tracking and sensing system, comprising:
directing a collimated laser beam from a laser along an optical transmit path, using at least a mirror coupled to a beam steering actuator;
directing return light towards collection optics along an optical return path using at least the beam steering actuator; and
directing the return light received by the collection optics towards a position-sensitive detector, the position-sensitive detector being operably coupled to a position tracking analyzer and an optical sensing analyzer, where information generated by said position-sensitive detector is configured to be used for retroreflector tracking and/or optical sensing.

19. The method of claim 18, further comprising controlling the beam steering actuator based on a determination by the position tracking analyzer.

20. The method of claim 18, further comprising:
extracting optical sensing data from the information generated by said position-sensitive detector with the optical sensing analyzer; and/or
extracting spectroscopic data from the information generated by said position-sensitive detector with the optical sensing analyzer.
